(19) United States
(12) Patent Application Publication
(10) Pub. No.: US 2023/0128019 A1
(43) Pub. Date: Apr. 27, 2023

(54) Dot Printing Method And Device For Additive Manufacturing Of Dosage Forms Which Contain Active Substances

(71) Applicant: DiHeSys Digital Health Systems GmbH, Schwäbisch-Gmünd (DE)

(72) Inventors: Markus Dachtler, Ulm (DE); Gerald Huber, Plüderhausen (DE)

(21) Appl. No.: 17/783,818

(22) PCT Filed: Dec. 2, 2020
(86) PCT No.: PCT/EP2020/084336

§ 371 Date: Dec. 14, 2022

(30) Foreign Application Priority Data
Dec. 9, 2019 (EP) 19214624.9

(51) Int. Cl. B29C 64/118; B29C 64/209; B33Y 10/00; B33Y 30/00; A23P 20/20; A23P 10/28; A23L 33/00

(52) U.S. Cl. CPC B29C 64/118; B29C 64/209; B33Y 10/00; B33Y 30/00; A23P 20/20; A23P 10/28; A23L 33/00; A23P 2020/253

(57) ABSTRACT

The present invention relates to a method and to a device for producing solid, at least semisolid dosage forms of pharmaceutical active agents, nutraceutical active agents and/or dietary supplemental active agents, wherein the dosage form is generated in an additive fashion by depositing a building substance in the form of dots.

### FIELD OF THE INVENTION

mon The present invention relates to a method and to a device for producing solid, at least semisolid, dosage forms of pharmaceutical active agents, nutraceutical active agents and/or dietary supplemental active agents, wherein the dosage form is generated in an additive fashion by depositing a building substance in the form of dots.

### BACKGROUND OF THE INVENTION

An additive 3D printing process for producing pharmaceutical dosage forms by means of fused filament fabrication (FFF) is known from WO 2016/038356 A1.

### SUMMARY OF THE INVENTION

The technical problem of the present invention is the provision of a method and a device for producing active agent-containing objects, especially active agent-containing dosage forms, having a particularly light and material-saving make up.

The above technical problem is solved by the embodiments of the present invention as disclosed in the claims and the present description.

In particular, the present invention provides a method for producing an at least semi-solid dosage form, the method comprising:
(i) providing a printer at least capable of 3D printing of the solid dosage form,
   the printer comprising
   a building platform on which the dosage form is printed,
   a printing head designed for applying an array of dots of a building substance for the dosage form on the building platform wherein the building substance is flowable, preferably through heating, when it is printed, and becomes at least semi-solid, preferably by cooling;
(ii) applying an array of dots of the building substance on the building platform wherein the dots can overlap or contact or not contact each other; or
(iii) at least semi-solidifying, preferably solidifying, of the dots applied in step (ii) such that the building substance becomes at least semi-solid;
(iv) applying a further array of dots on the previous dots in such a manner that the dots of the further array overlap with the dots of the previous array; and
(v) repeating steps (ii) to (iv)) until the dosage form is built;
   wherein the building substance contains at least one pharmaceutical active agent and/or at least one nutraceutical active agent and/or at least one dietary supplemental active agent.

### DETAILED DESCRIPTION OF THE INVENTION

A “dot” according to the invention is an essentially round, per se three-dimensional structure obtained by impaction of the volume unit of the building substance which is dispensed from a printing head of the printing device in liquid, at least flowable form typically having the shape of a drop, of a (approximated) rotation ellipsoid or of a (approximated) sphere, and deposited on the building platform (in step (ii)) or, at least partially, on the previously deposited dots (in step(s) (iii)).

According to the invention, the building substance contains at least one active agent which may be selected from pharmaceutical, nutraceutical and dietary supplemental active agents. In the following these active agents are also collectively denoted as “active component(s)” or also as “active agent(s)”.

The active components can be selected essentially from any available pharmaceutical, nutraceutical and dietary supplemental active agents which can be administered by means of semi-solid or solid dosage forms, which active agents can also be combined as long as this is acceptable for the intended application. Preferred dosage forms are manufactured by using building substances containing at least one pharmaceutical active agent. Preferred dosage forms according to the inventrion are synergistic combinations of 2 or more pharmaceutical active agents which can be present in a single building substance. Alternatively, different active agents can be present in different building substances forming the applied dots. Thus, according to the invention, an active agent can be present in a building substance forming a first group of dots and one (or more) different active agent(s) can be contained in a different group of dots (i.e. at least two building substances are present containing the active agent or the respective active agents, respectively). Preferably, the different active agents are contained in different building substances. In this embodiment it is possible that the respective building substances are the same except for the respective active agent(s), or they can be different for example, for providing characteristic and tailor-made features depending on the active agent such as for example, pH value, solubility, consistency, ion milieu, particle size, color etc. In certain embodiments of the invention combinations of 2 or more pharmaceutical active agents are provided for example, wherein a pharmaceutical active agent is contained for a certain indication and a further pharmaceutical active agent is present in the same or a different building substance for example, at least reducing, optimally suppressing a side effect at least potentially exerted by the first active agent, which is a particularly preferred embodiment of the invention.

Examples of pharmaceutical active agents which may be combined according to the invention include agonists/antagonists for reducing the addictive potential of analgesics (e.g., Tilidine and Naloxone), combination preparations for treatment of for example, high blood pressure (preferably combinations of ACE inhibitor and/or calcium channel blocker and/or beta-blocker), allergies (e.g. antihistamines and Calcium) diarrheas (e.g. minerals and Loperamide), stroke/prophylaxis of infarction (anticoagulants and/or antihypertensives), Parkinson's disease (Levodopa and Benserazide), hypercholesterolemia (e.g. Statins and/or fish oils), deficiency conditions (e.g. combinations of vitamins and minerals) and analgesics (e.g. an analgesics and caffeine) etc.

According to preferred embodiments of the invention, dots having the same active agent or having the same combination of active agents or having the same concentration of active agent(s) are present in a common section of the dosage form such that the dots in the same group at least partially adjoin one another at least one side. Thus, the dots having the same active agent or having the same combination of active agents or having the same concentration of active agent(s) form at least one common section such as for example, at least one common layer or at least one contiguous part of a layer, which can be oriented, with respect to the longest dimension of the dosage form, horizontally or vertically. In other embodiments, dots having the same active agent or having the same combination of active agents or having the same concentration of active agent(s) can be grouped in several sections (e.g., 2 or more layers and/or partial layers). Such sections can also have different release properties for the active agent(s) such as for example, different pH conditions, solubility, gastric juice resistance, other solubility behaviors (e.g. in that dots of particular sections or of one particular section contain a burst release substance wherein one method for providing a burst release embodiment is preferably designed by applying the dots of the burst release sections in such a manner that the burst release sections envelope the sections of spots not having the burst release agent in their building substance, i.e. at least one, preferably several layers of dots containing burst release substance(s) in the building substance(s) surround the sections not having burst release function in every dimension in the dosage form).

Preferably, the printer comprises at least one printing head connected to a reservoir containing the building substance so that the at least one printing head is capable of withdrawing an amount of the building substance for applying the building substance in steps (ii) to (v). The reservoir can be embodied in different ways depending on the kind and consistency of the building substance. Thus, in the case of liquid substances, the reservoir may be a container for liquids being in contact with the printing head via a line for the building substance through which the building substance is transported, typically pumped, to the printing head. In other embodiments, the building substance can be present in the reservoir in solid or semi-solid form e.g., as a powder or as granules, wherein a conveying mechanism supplies the solid or semi-solid building substance to the printing head. In this embodiment the printing head typically comprises a heating or melting device which transforms the building substance into an at least flowable, preferably liquid, form which is then deposited, i.e. printed, typically by depositing means, on the building platform.

Alternatively, the building substance can also be present in form a solid or at least semi-solid filament wherein the filament can be present e.g., in a supply funnel or supply tube forming the reservoir. Such reservoir types can be designed in various ways whereby in the case of completely solid filament building substances linear embodiments are most frequently chosen. Preferred filament building substances are mostly elongated, cylindric objects being typically more or less flexible, therefor being amenable to be receivable in bent reservoirs such as reservoir coils and being supplied to the printing head by pushing or pressing mechanisms. In case the elasticity of the filament is insufficient for being supplied in spiral form by reservoir coils the filament can also supplied in the form of short, linear sticks from a reservoir cartridge.

In the case of liquid building substances, the printing head can comprise piezo-electric means for dispensing the volume increment such that the building substance is dispensed in a manner like that of an ink jet printer. Such an embodiment can also be designed as a 2D printing process as will be further outlined below. A 2D printing process comprises the application of a liquid (examples are indicated below) typically through known techniques such as piezo-electric dispensing means e.g., at least on a section of the surface of a dosage form prepared by 3D printing through the above-described steps such as in a further step (vi), wherein the liquid is fixed on the at least one rection of the surface by drying or in a different way (e.g., chemically and/or by light radiation which is typically realized by a laser device). It is, of course, also possible to apply one or more 2D-printed layers within the dosage form and to continue with the 3D printing steps after application of a 2D layer (wherein it is, of course, possible that a final 2D-printed layer can follow).

In a further preferred embodiment, the printer comprises more than one printing head, wherein 2 to 10 printing heads are particularly preferred. Embodiments of the invention using several printing heads can serve different functions: in one embodiment it is foreseen that a dosage form is printed using more than one printing head e.g., to print different building substances having different features as outlined above. In one embodiment employing more than one printer, 3D and 2D printing heads can be used wherein it is also possible, as will be outlined below, that printing heads for use in the invention can be designed for 3D printing as well as for 2D printing. It is furthermore envisaged to use more than one printing head for printing of several dosage forms simultaneously. Of course, it is also possible according to the invention to simultaneously print several different dosage forms i.e., so to speak sets of printing heads are formed or at least addressed as sets printing simultaneously several dosage forms using different building substances, wherein the simultaneously printed dosage forms can have the same of different make-ups. The number of printing heads can also substantially extend above 10 printing heads for being able to correspondingly increase the number of simultaneously printed dosage forms. In the context of the embodiment using more than one printing head it is further preferred that each of the printing heads is connected to a reservoir containing the building substance so that the respective printing head is capable of withdrawing an amount of the building substance for applying the building substance in steps (ii) to (v). In this embodiment, the forms of the reservoirs can be the same or different.

The generation of the dots is thus carried out according to the invention by applying a volume increment of the building substance wherein the volume increment preferably has a volume of 20 pl to 30 μl. If needed, several volume increments can be applied successively.

According to a further preferred embodiment the printing head or, in the case of several printing heads, at least one of the printing heads, respectively, is designed for 2D printing as well as 3D printing. In another embodiment of the invention, the device can have at least one or more 3D printing heads, and one 2D printing head, if needed. 3D printing heads are designed for applying semi-solid and molten building substances whereas 2D printing heads are designed for applying building substances being already in liquid form without heating in the printing head such as e.g., inks or active agent solutions, active agent emulsions and active agent suspensions.

In a preferred embodiment, the dots are applied in a manner such that the dots applied in step (ii) and the further steps (ii) (according to step (v) of the method according to the invention) overlap with the dots applied in the previous step (step (ii) or every step (ii) of the further building steps according to step (v)). This embodiment results in a dosage form forming, in the sense of a brick arrangement of the applied dots, a particularly stable arrangement, if the dots of a layer show a complete overlap with the previous layer. On other hand, application of a layer of dots partially overlapping with the previous layer provides a particularly lightweight arrangement having gaps. In this way an enlarged surface of the dosage form being exposed to the surrounding milieu is created through which the solubility or degradation rate in the surrounding milieu, especially the intestinal tract of the subject, such as a human patient, taking the dosage form, can be regulated.

As described above, the building substance is present in preferred embodiments as a filament and the printing head is, in the case of 3D printing, designed for melting an amount of the filament, preferably in that the printing head, as described above, comprises a heating device for applying the array of dots of the building substance on the building platform in step (ii) as well as the further array(s) of dots on the previous array of dots in step (iv).

The solidification (at least to a semi-solid state) of the printed building substance as well as the bonding between the applied individual dots in step (iii) can be accomplished in various ways. In one embodiment employing a meltable material the bonding between such dots by solidification after application on the carrier structure wherein the solidification may be carried out by various mechanisms such as simple cooling and/or chemically through known substances. In a different embodiment, a suitable binder can be added to the building substance such as a dispersion or a solution, wherein the binder provides for curing of the building substance after application of the dot, the curing through the binder may occur e.g., through heat which may be applied via a suitable heat source in the printing device such as a light source, preferably a laser device. Curing exerted by the binder can also occur chemically by corresponding starter molecules and/or by light of a suitable wavelength wherein the latter is preferably emitted by means of a laser device. In a further embodiment, the building substance can contain one or more starting compounds, typically monomers, of one or more polymers, and after application of the dot(s) a polymerization is started by suitable means such as, again, light, heat, or other polymerization starters, whereby the respective applied dot is cured and bonded or adhered, respectively, to adjacent dots.

Suitable carrier materials being flowable at the printing temperature in which the active agent(s) are present are e.g., in general carriers suitable for hot melt extrusion (HME) such as low melting waxes and polymers. The HME mixture or in general the building substance mixture, respectively, can contain, besides the low melting carrier, further process agents and aids such as binders, emollients, antioxidants, perfumes, sweeteners or similar. Suitable HME carriers and emollients are known to the skilled person and disclosed e.g., in Crowley et al. (2007) Drug Development and Industrial Pharmacy, 33, 909-926 (carriers: pages 917 to 919, in particular Table 1; emollients: pages 917 and 920, in particular Table 2) wherein it is expressis verbis referred to the indicated passages in the present disclosure.

The present additive 3D printing method and the present combined additive 3D and 2D printing method, respectively, is preferably carried out computer aided. Thus, typically in pre-step before step (i), a calculated two- or three-dimensional image of the object to be printed is calculated e.g., by means of a known CAD program. The computer-generated representation of the object to be printed can also be made by scanning of an existing object e.g., a pharmaceutical dosage form or a medical device. During the present method, the computer-generated image model is then subdivided into the desired spots wherein the resolution of the real object is higher the smaller the volume increments of the building substance for the generated dots are. For example, an active agent, a carrier substance or carrier compositions, respectively, and/or further aids such as color substances, pH dependently soluble substances, temperature-dependently melting substances and other optionally required materials as well as their amounts (concentration in the volume increment deposited for the application of the dots) can be assigned to each individual dot and finally be printed. As regards, suitable components for typical printing devices it is referred to e.g., US 2017/0368755 A1 and U.S. Pat. No. 6,070,107 B2.

Objects containing active agents printable by the method are especially semi-solid or solid pharmaceutical dosage forms such as e.g., tablets, capsules, implants, plasters, suppositories, pellets, printed granules, or oral and topical, respectively, thin films. Tablets producible by the method according to the invention are manifold and include e.g., oblong shape tablets, lozenges, implantable tablets, multiapplication tablets, disperse tablets, retard tablets, vaginal tablets, eye tablets, coated tablets, matrix tablets, chewable tablets, film-coated tablets, modified-release tablets, lacquer tablets and enteric coated tablets.

Further particularly suitable objects are medical devices such as active agent-containing topical drug forms, contact lenses, plasters, which preferably release the active agent(s) for topical application. In one embodiment, the method of the present invention is carried out under sterile conditions. By means of this embodiment, implants are also provided according to the invention. Further active agent-containing object provided under sterile conditions are injectable objects.

As already disclosed at the outset the present invention relates under a further aspect to a device for producing a solid dosage form, especially for carrying out the method according to the invention, the device comprising:
(a) a building platform on which the dosage form is printed;
(b) at least one printing head designed for applying an array of dots of a building substance for the dosage form on the building platform, wherein the building substance is flowable during the printing state, and becomes at least semi-solid, preferably solid;
   wherein the building substance contains at least one pharmaceutical active agent and/or at least one nutraceutical active agent and/or at least one dietary supplemental active agent.

Preferred embodiments of the device have already been outlined in the context of the disclosure of the method according to the invention.

1. A method for producing a solid dosage form, the method comprising:
(i) providing a printer at least capable of 3D printing of the solid dosage form, the printer comprising
a building platform on which the dosage form is printed,
a printing head designed for applying an array of dots of a building substance for the dosage form on the building platform wherein the building substance is flowable, preferably through heating, when it is printed, and becomes at least semisolid, preferably by cooling;
(ii) applying an array of dots of the building substance on the building platform wherein the dots may overlap or contact or not contact each other; or
(iii) at least semi-solidifying, preferably solidifying, of the dots applied in step (ii) in such that the building substance becomes at least semi-solid;
(iv) applying a further array of dots on the previous dots in such a manner that the dots of the further array overlap with the dots of the previous array; and
(v) repeating steps (ii) to (iv) until the dosage form is built;
wherein the building substance contains at least one pharmaceutical active agent and/or at least one nutraceutical active agent and/or at least one dietary supplemental active agent.

2. The method of claim 1 wherein the printer comprises at least one printing head connected to a reservoir containing the building substance so that the at least one printing head is capable of withdrawing an amount of the building substance for applying the building substance in steps (ii) to (v).

3. The method of claim 2 wherein the printer comprises more than one, preferably 2 to 10 or more, printing head(s).

4. The method of claim 3 wherein each of the printing heads is connected to a reservoir containing the building substance so that the respective printing head is capable of withdrawing an amount of the building substance for applying the building substance in steps (ii) to (v).

5. The method of claim 1 wherein the building substance is present in the form of a filament and the printing head is designed to melt an amount of the filament for applying the array of dots on the building platform in step (ii) and the further array of dots on the previous array of dots in step (iv).

6. The method according to claim 1 wherein the dots are generated by applying a volume increment of the building substance and the volume increments have a volume of 20 pl to 30 μl.

7. A device for producing a solid dosage form, the device comprising:
(a) a building platform on which the dosage form is printed;
(b) at least one printing head designed for applying an array of dots of a building substance for the dosage form on the building platform, wherein the building substance is flowable, preferably when it is in a heated state, and becomes at least semi-solid, preferably solid, by solidification, preferably by cooling;
wherein the building substance contains at least one pharmaceutical active agent and/or at least one nutraceutical active agent and/or at least one dietary supplemental active agent.

8. The device of claim 7 wherein the printer comprises a reservoir containing the building substance, the reservoir being connected to the printing head so that same is capable of withdrawing an amount of the building substance for applying the building substance on the building platform.

9. The device of claim 7 wherein the at least one printing head is designed for 2D and 3D printing.
